(19) United States
(12) Patent Application Publication
(10) Pub. No.: US 2011/0075909 A1
(43) Pub. Date: Mar. 31, 2011

(54) X-ray imaging system, imaging method and computer readable media including imaging program

(75) Inventors: Eiichi Kanagawa, Kanagawa (JP); Noriaki Ida, Kanagawa (JP); Sadato Akahori, Kanagawa (JP); Yasunori Ohta, Kanagawa (JP)

(73) Assignee: FUJIFILM Corporation, Tokyo (JP)

(21) Appl. No.: 12/923,610

(22) Filed: Sep. 29, 2010

(30) Foreign Application Priority Data
Sep. 30, 2009 (JP) 2009-227976

(51) Int. Cl. G06T 7/00; A61B 6/03

(52) U.S. Cl. 382/131; 378/4

(57) ABSTRACT

The X-ray imaging system includes an imager in which projection data of X-ray images taken at different angles is acquired in tomosynthesis imaging, a first image reconstruction unit in which the acquired projection data of the X-ray images is used to reconstruct an X-ray tomographic image, an abnormal pixel data identification unit which refers to a defective pixel map in which information on defective pixels due to a flat panel detector and its peripheral circuit has been stored, to identify, from among pixel data of the reconstructed X-ray tomographic image, pixel data of abnormal pixels corresponding to the defective pixels, and a first image correction unit which corrects the identified pixel data of the abnormal pixels of the X-ray tomographic image to output a first X-ray tomographic image.

### BACKGROUND OF THE INVENTION

The present invention relates to an X-ray imaging system, an imaging method, and a computer readable media including an imaging program for reconstructing an X-ray tomographic image at a cross section located at a given height of a subject using projection data of X-ray images acquired by tomosynthesis imaging.

An X-ray imaging system for tomosynthesis imaging irradiates a subject with X-ray at different angles while moving an X-ray source in one direction and detects the X-ray with which the subject has been irradiated with a flat panel X-ray detector (FPD) to achieve acquisition of projection data corresponding to X-ray images of the subject taken at the different angles by a single imaging operation. Then the process proceeds to reconstruction processing using the projection data of the acquired X-ray images to acquire an X-ray tomographic image at a cross section of the subject at a given height thereof.

Now, reconstruction of an X-ray tomographic image will be described.

In tomosynthesis imaging, an X-ray source starts to move from the position S1 to the position S3 and a subject 30 is irradiated with X-ray from the positions S1, S2, and S3 to acquire X-ray images (projection data) P1, P2, and P3 of the subject 30 as illustrated in FIG. 5A.

Now considering the case where imaging objects A and B are present at two positions different in height of the subject 30 as illustrated in FIG. 5A, the X-ray emitted from the imaging positions S1, S2 and S3 by the X-ray source passes through the subject 30 to enter an FPD. As a result, the two imaging objects A, B are projected in different positional relationships on the X-ray images P1, P2 and P3 corresponding to the imaging positions S1, S2 and S3.

In the X-ray image P1, for example, the X-ray source, located in the position S1 to the left of the imaging objects A, B with respect to the direction of movement of the X-ray source, causes projections of the imaging objects A, B to be formed in positions P1A, P1B that are set off to the right of the imaging objects A, B. Likewise, in the X-ray image P2, the projections are formed in positions P2A, P2B that are substantially directly beneath the imaging objects A, B; in the X-ray image P3, the projections are formed in positions P3A, P3B that are set off to the left of the imaging objects A, B.

To reconstruct an X-ray tomographic image of the subject at a cross section located at the height of the object A, the X-ray image P1 is shifted leftward, and the X-ray image P3 is shifted rightward, for example, so that the projection positions P1A, P2A, and P3A of the object A coincide with each other as illustrated in FIG. 5B (shift addition method). Thus, an X-ray tomographic image is reconstructed at the height of the object A. An X-ray tomographic image at a cross section located at a given height can also be reconstructed in the same way.

The FPD comprises photoelectric conversion elements corresponding to the respective pixels of an X-ray image arranged in matrix form. However, there are a pixel at which X-ray cannot be detected, and a pixel having a different X-ray detection sensitivity from other pixels mainly because of problems in manufacturing technology and the peripheral circuit. Such a pixel of an image produced from the pixel of the FPD is hereinafter referred to as “defective pixel”. In order to reduce or eliminate adverse effects of the defective pixels on the image, the defective pixels on the FPD panel are kept in mind beforehand and various corrections are performed on the projection data read out of the FPD.

JP 2007-632 A, for example, relates to compensation of an offset signal produced by a flat panel detector of a radiographic imaging apparatus. The literature describes an offset compensation for the flat panel detector using an offset map.

### SUMMARY OF THE INVENTION

As described above, however, tomosynthesis imaging acquires, during a single imaging operation, projection data corresponding to a plurality of X-ray images, each of which has a large size, resulting in projection data having a large data quantity. Accordingly, where each and every image data having a large data quantity such as projection data of X-ray images as acquired in tomosynthesis imaging was corrected as proposed in JP 2007-632 A, reconstruction of an X-ray tomographic image required a long time.

An object of the present invention is to solve the above problems associated with the prior art and provide an X-ray imaging system, an imaging method and a computer readable media including an imaging program capable of reconstructing an X-ray tomographic image by tomosynthesis imaging at a high speed within a short time period.

In order to achieve the above object, the present invention provides an X-ray imaging system comprising:
an imager in which a subject is irradiated with X-ray at different angles while moving an X-ray source in one direction and the X-ray with which the subject has been irradiated is detected with a flat panel detector to acquire projection data of X-ray images taken at the different angles in tomosynthesis imaging;
a first image reconstruction unit in which the projection data of the X-ray images acquired by the imager is used to reconstruct an X-ray tomographic image;
an abnormal pixel data identification unit which refers to a defective pixel map in which information on defective pixels due to the flat panel detector and its peripheral circuit has been stored, to thereby identify, from among pixel data of the X-ray tomographic image reconstructed by the first image reconstruction unit, pixel data of abnormal pixels corresponding to the defective pixels; and
a first image correction unit which corrects the pixel data of the abnormal pixels of the X-ray tomographic image identified by the abnormal pixel data identification unit to output a first X-ray tomographic image.

The present invention also provides an X-ray imaging method comprising the steps of:
irradiating a subject with X-ray at different angles while moving an X-ray source in one direction, and detecting the X-ray with which the subject has been irradiated with a flat panel detector to acquire projection data of X-ray images taken at the different angles in tomosynthesis imaging;
performing a first reconstruction in which the projection data of the acquired X-ray images is used to reconstruct an X-ray tomographic image;
performing a first identification for identifying, from among pixel data of the X-ray tomographic image reconstructed by the first image reconstruction, pixel data of abnormal pixels corresponding to defective pixels of the flat panel detector by reference to a defective pixel map in which information on the defective pixels due to the flat panel detector and its peripheral circuit has been stored; and
performing a first correction for correcting the pixel data of the abnormal pixels of the X-ray tomographic image identified by the first identification to output a first X-ray tomographic image.

The present invention further provides a computer readable media including an X-ray imaging program for causing a computer to execute:
a step of receiving projection data of X-ray images taken at the different angles acquired by irradiating a subject with X-ray at different angles while moving an X-ray source in one direction and detecting the X-ray with which the subject has been irradiated with a flat panel detector in tomosynthesis imaging;
a step of performing a first reconstruction for reconstructing an X-ray tomographic image using the received projection data of the X-ray images;
a step of performing a first identification for identifying, from among pixel data of the X-ray tomographic image reconstructed by the first reconstruction, pixel data of abnormal pixels corresponding to defective pixels of the flat panel detector by reference to a defective pixel map in which information on the defective pixels due to the flat panel detector and its peripheral circuit has been stored; and
a step of subjecting the pixel data of the abnormal pixels of the X-ray tomographic image identified by the first identification to a first correction to output a first X-ray tomographic image.

The present invention reconstructs an X-ray tomographic image, identifies pixel data of abnormal pixels of the X-ray tomographic image corresponding to defective pixels on the FPD and corrects the identified pixel data of the abnormal pixels of the X-ray tomographic image and therefore the reconstructed X-ray tomographic image can be acquired at a high speed in a short time period.

### BRIEF DESCRIPTION OF THE DRAWINGS

FIG. 1 is a block diagram representing a configuration of an X-ray imaging system according to an embodiment of the invention.

FIG. 2 is a block diagram illustrating a detailed configuration of an image processor in the imaging system shown in FIG. 1.

FIG. 3 is a conceptual view illustrating how tomosynthesis imaging is made in the imaging system shown in FIG. 1.

FIGS. 4A and 4B are conceptual views illustrating a positional relationship between a defective pixel on an FPD and an abnormal pixel of an X-ray tomographic image which linearly extends along the direction of movement of an X-ray source under the influence of the defective pixel.

FIGS. 5A and 5B are conceptual views illustrating reconstruction of an X-ray tomographic image in tomosynthesis imaging.

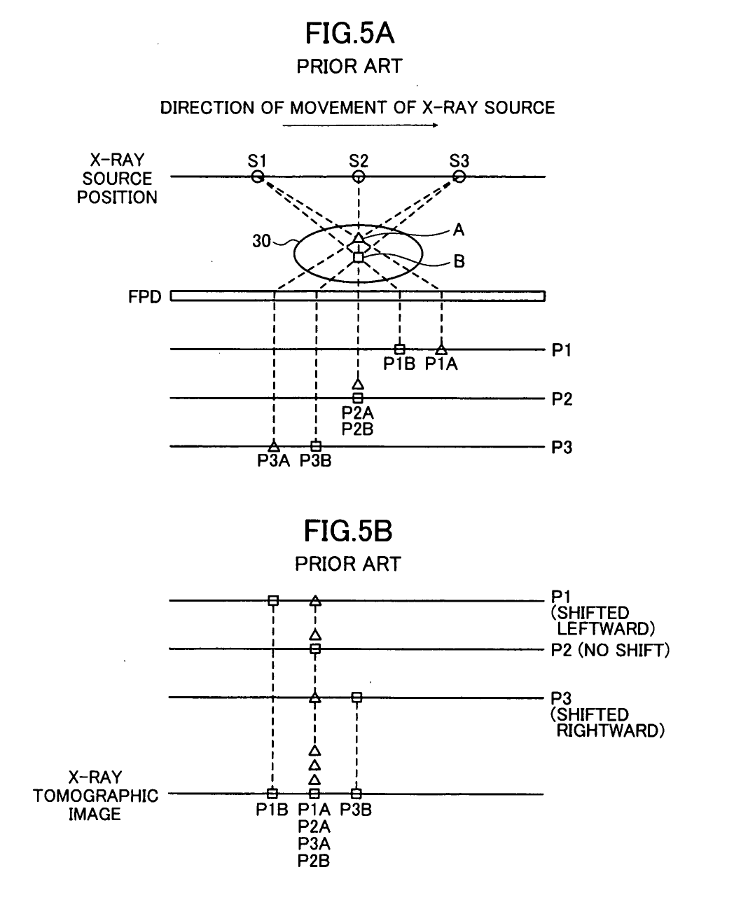

### DETAILED DESCRIPTION OF THE INVENTION

The X-ray imaging system, imaging method, and a computer readable media including an imaging program of the invention will be described in detail with reference to the preferred embodiments shown in the accompanying drawings.

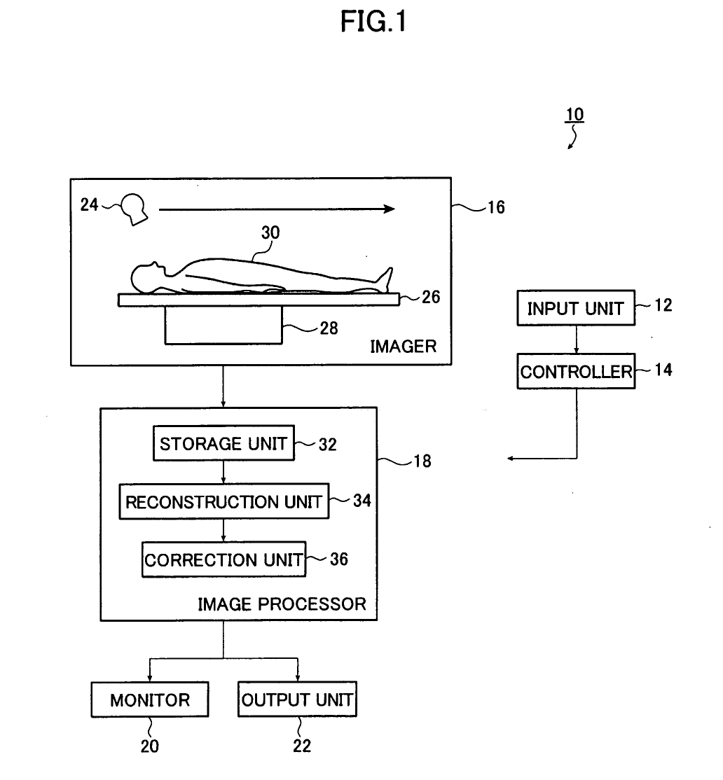

FIG. 1 is a block diagram representing a configuration of an X-ray imaging system according to an embodiment of the invention. An X-ray imaging system 10 shown in FIG. 1 acquires images of a subject 30 such as a human body by tomosynthesis imaging (X-ray imaging) and reconstructs an X-ray tomographic image at a cross section located at a given height of the subject 30. The imaging system 10 comprises an input unit 12, a controller 14, an imager 16, an image processor 18, a monitor 20, and an output unit 22.

The input unit 12 is provided to enter various instructions including an instruction to start imaging and an instruction to switch operations described later and may include a mouse and a keyboard. The input unit 12 produces an instruction signal, which is received by the controller 14.

The controller 14 produces control signals according to an instruction signal transmitted from the input unit 12 to control the operations of the imaging system 10 including imaging operations by the imager 16, image processing by the image processor 18, screen display by the monitor 20, and output processing by the output unit 22. Although not shown, the control signals outputted from the controller 14 are received by the imager 16, the image processor 18, the monitor 20 and the output unit 22.

The imager 16 acquires images of the subject 30 by tomosynthesis imaging according to a control signal supplied from the controller 14; it comprises an X-ray source 24, a carrier (not shown) for moving the X-ray source 24, an imaging table 26, and a flat panel type X-ray detector (FPD) 28.

The X-ray source 24 is spaced apart by a predetermined distance above the subject 30 that is disposed on the top surface of the imaging table 26.

The FPD 28 is disposed on the bottom side of the imaging table 26 so that its X-ray receiving surface faces upwards. The FPD 28 detects X-ray having passed through the subject 30 and effects photoelectric conversion to produce digital image data (projection data) corresponding to an acquired X-ray image of the subject 30. The FPD 28 may be of a direct type in which radiation is directly converted into an electric charge, an indirect type in which radiation is temporarily converted into light, which is then converted into an electric signal, or any of various other types. The FPD 28 may be configured so that it is movable in the same direction as the X-ray source 24.

In tomosynthesis imaging, the carrier controls the movement of the X-ray source 24 to move the X-ray source 24 in one direction and change the X-ray irradiation angle toward the subject 30 so as to irradiate the subject 30 with X-ray at different imaging angles (i.e., at given time intervals). The X-ray emitted by the X-ray source 24 passes through the subject 30 to enter the light receiving surface of the FPD 28, and the FPD 28 detects the X-ray and converts the detected X-ray into electricity to obtain projection data corresponding to the acquired X-ray image of the subject 30.

In tomosynthesis imaging, a plurality of X-ray images, for example, 20 to 80 images, of the subject 30 having at different imaging angles are taken by a single imaging operation, whereupon the FPD 28 sequentially outputs projection data corresponding to the acquired X-ray images.

Subsequently, the image processor 18 receives the projection data of the X-ray images acquired by the imager 16 according to the control signal supplied from the controller 14 and performs image processing (including correction and image reconstruction) using the projection data of the X-ray images to reconstruct an X-ray tomographic image of the subject 30 at a cross section thereof located at a given height. The image processor 18 comprises a storage unit 32, a reconstruction unit 34 and a correction unit 36.

The storage unit 32 stores the projection data of the X-ray images acquired by the imager 16. The reconstruction unit 34 uses the projection data of the X-ray images stored in the storage unit 32 to perform reconstruction processing, thus forming an X-ray tomographic image of the subject 30 at a cross section thereof at a given height. The correction unit 36 performs given corrections to the X-ray tomographic image reconstructed by the reconstruction. unit 34. The image processor 18 will be described later in detail.

The image processor 18 may be configured by hardware (a device) or a program for causing a computer to execute a part of the X-ray image processing method of the invention.

Subsequently, the monitor 20 displays an X-ray tomographic image reconstructed by the image processor 18 according to the control signal supplied from the controller 14 and may be exemplified by a flat panel display such as a liquid crystal display.

The output unit 22 outputs an X-ray tomographic image reconstructed by the image processor 18 according to the control signal supplied from the controller 14 and may be exemplified by a thermal printer for printing out the X-ray tomographic image and a storage device for storing digital image data of the X-ray tomographic image in any of various recording media.

Next, the image processor 18 will be described in detail.

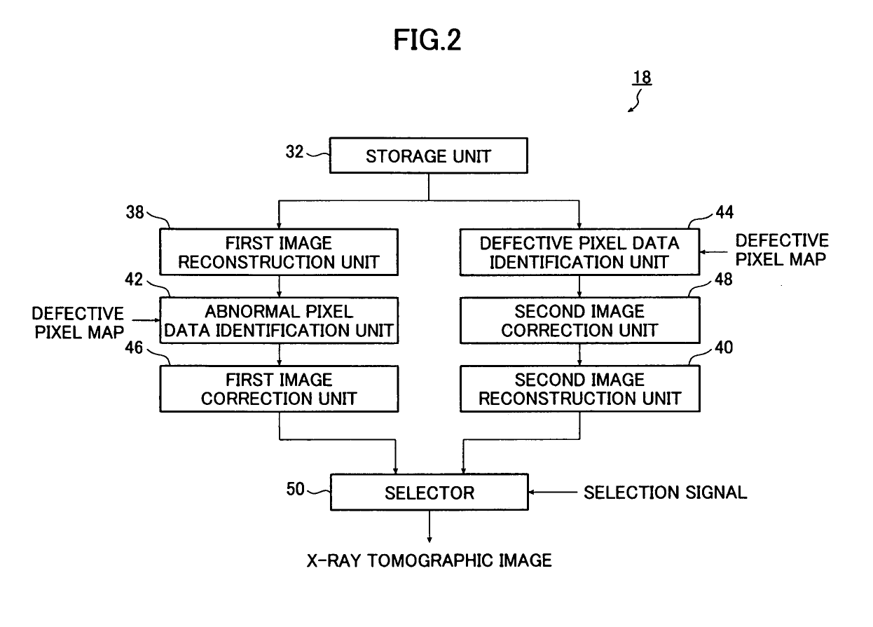

FIG. 2 is a block diagram illustrating a detailed configuration of the image processor shown in FIG. 1. As shown in FIG. 2, the image processor 18 comprises the storage unit 32, first and second image reconstruction units 38, 40, an abnormal pixel data identification unit 42, a defective pixel data identification unit 44, first and second image correction units 46, 48, and a selector 50. The abnormal pixel data identification unit 42, the defective pixel data identification unit 44, and the first and second image correction units 46, 48 correspond to the correction unit 36, and the first and second image reconstruction units 38, 40, and the selector 50 correspond to the reconstruction unit 34.

The first image reconstruction unit 38 uses projection data of X-ray images acquired by the imager 16 by taking at different angles and stored in the storage unit 32 to perform reconstruction processing to thereby form an X-ray tomographic image at a cross section located at a given height of the subject 30.

The abnormal pixel data identification unit 42 refers to a defective pixel map to identify, from among pixel data of the X-ray tomographic image reconstructed by the first image reconstruction unit 38, pixel data of an abnormal pixel (abnormal pixel data) corresponding to a defective pixel on the FPD 28.

Information on defective pixels due to the FPD 28 and its peripheral circuit (including positional information and information on the state of the defective pixels) is stored in the defective pixel map. The defective pixel map may be produced, for example, based on projection data of an X-ray image taken in the absence of the subject 30. The defective pixel map is stored in, for example, the FPD 28 or the controller 14.

The defective pixel is a pixel, as described above, incapable of outputting pixel data proportional to the received

X-ray dosage such as a normal pixel, and it includes a pixel at which X-ray cannot be detected because of, for example, a problem in manufacturing technology and a pixel having a different X-ray detection sensitivity from other normal pixels. Therefore, one defective pixel map may be used or a plurality of different defective pixel maps may be used according to the state of the defective pixels such as the pixel defect and faulty gain to perform corrections to be described later.

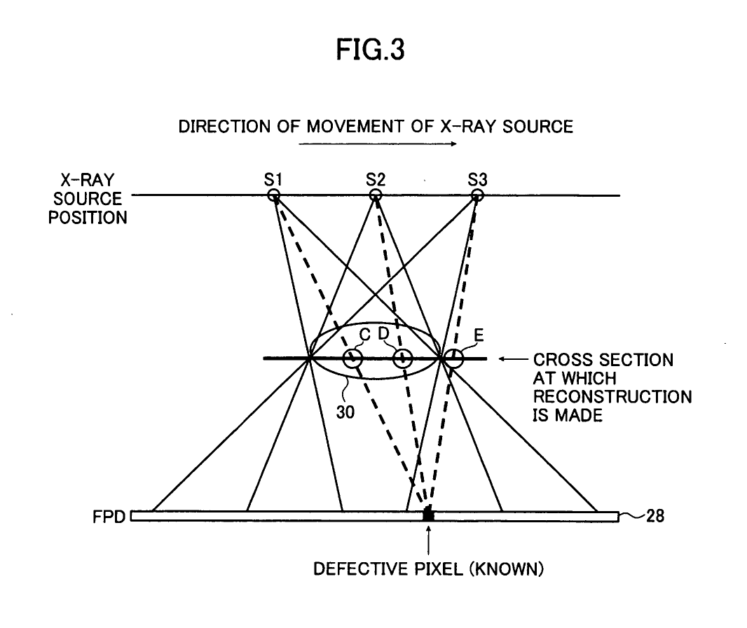

A specific method of identifying an abnormal pixel in the abnormal pixel data identification unit will be described below by reference to FIG. 3. It is assumed that a defective pixel known from the defective pixel map is present at a given position on the FPD 28 as shown in FIG. 3. In tomosynthesis imaging, the X-ray source is moved in one direction to irradiate the subject 30 with X-ray from positions S1, S2 and S3. The influence of the defective pixel known from the defective pixel map appears on C when imaging is made from X-ray source position S1, on D when imaging is made from X-ray source position S2, and on E when imaging is made from X-ray source position S3. The abnormal pixel can be geometrically determined from the distance (l) between the X-ray source and the FPD 28, the positions (m) of the X-ray source and the tomographic image to be reconstructed, and the position of the defective pixel on the FPD 28. The values of l and m may be fixed or known for each imaging system, or be easily obtained from a sensor (not shown) attached to the system.

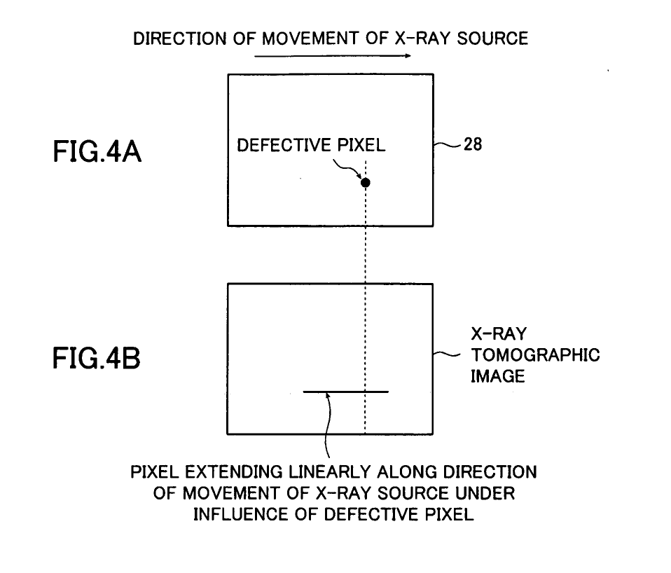

When the FPD 28 contains one defective pixel as shown in FIG. 4A, the X-ray tomographic image reconstructed by the first image reconstruction unit 38 has an abnormal pixel which linearly extends along the direction of movement of the X-ray source 24 under the influence of the defective pixel as shown in FIG. 4B. The position of the defective pixel on the FPD 28 is different from the position and the shape of the abnormal pixel on the X-ray tomographic image under the influence of the defective pixel. Even in such a case, the position of the abnormal pixel on the X-ray tomographic image under the influence of the defective pixel can be geometrically identified from the position of the defective pixel in the same manner as above.

Subsequently, the first image correction unit 46 uses the reconstructed X-ray tomographic image to correct the pixel data (pixel value) of the abnormal pixel of the X-ray tomographic image identified by the abnormal pixel data identification unit 42 and outputs a first X-ray tomographic image.

The abnormal pixel data may be corrected by a method in which the abnormal pixel is regarded as a defective pixel and is corrected by using pixel values of normal pixels in the vicinity of the abnormal pixel on the reconstructed X-ray tomographic image (application of the method described in JP 2007-632 A). Such corrections may be implemented using various methods including known methods. In addition, if it is known that abnormal pixel data appears linearly as already described above, methods disclosed in JP 2007-632 A and JP 2008-018047 A may be used to correct the linear abnormal pixel.

On the other hand, the defective pixel data identification unit 44 refers to the defective pixel map to identify, from among the pixel data making up the projection data of the X-ray images taken at different angles as stored in the storage unit 32, the pixel data corresponding to the defective pixel on the FPD 28. The second image correction unit 48 corrects the X-ray images for the pixel data corresponding to the defective pixel identified by the defective pixel data identification unit 44 by interpolation using the pixel data corresponding to the normal pixels on the periphery of the defective pixel (e.g., 8 neighboring pixels surrounding the defective pixel). Such corrections may be implemented using various methods including known methods.

The second image correction unit 48 performs various corrections including offset correction, residual image correction, gain correction, defective pixel correction, step correction, longitudinal inconsistent density correction, and lateral inconsistent density correction for each of the X-ray images. The offset correction, residual image correction, gain correction, defective pixel correction, step correction, longitudinal inconsistent density correction, and lateral inconsistent density correction are known corrections and may be implemented each using any of various methods including known methods as described in, for example, JP 2007-632 A.

The second image reconstruction unit 40 uses the projection data of the X-ray images corrected by the second image correction unit 48 to perform reconstruction processing to thereby reconstruct a second X-ray tomographic image at the cross section located at the same height of the subject 30 as does the first image reconstruction unit 38.

The first X-ray tomographic image is corrected after its reconstruction and may therefore require less time for reconstruction than the second X-ray tomographic image. When 80 X-ray images are used to reconstruct an X-ray tomographic image, the first X-ray tomographic image is obtained more rapidly than the second X-ray tomographic image by the time required for identifying in the defective pixel data identification unit 44 the image data of the 80 X-ray images corresponding to the defective pixel and the time required to correct the X-ray images in the second image correction unit 48.

On the other hand, the second X-ray tomographic image is reconstructed after correction of the pixel data corresponding to the defective pixel using the pixel data corresponding to the normal pixels on the periphery of the defective pixel on the FPD 28 and therefore requires more time for the reconstruction than the first X-ray tomographic image but is of a higher quality.

Subsequently, the selector 50 outputs an X-ray tomographic image as it switches between the first X-ray tomographic image and the second X-ray tomographic image at a given timing according to the selection signal (control signal) supplied from the controller 14.

The defective pixel data identification unit 44, the second image correction unit 48, the second image reconstruction unit 40 and the selector 50 are not essential components of the image processor 18. These components are preferably provided as necessary when the output of the second X-ray tomographic image is required.

A third image correction unit may be provided between the storage unit 32 and the first image reconstruction unit 38 so that the respective X-ray images stored in the storage unit 32 are subjected to various simple corrections at a higher speed than in the second image correction unit before the first image reconstruction unit 38 reconstructs the X-ray tomographic image.

The timing at which selection is made in the selector 50 between the first X-ray tomographic image and the second X-ray tomographic image according to the selection signal is not limited in any manner, permitting use of various timings.

For example, the timing may be so set that the first X-ray tomographic image is outputted from the selector 50 after the first image correction unit 46 has completed correction of the pixel data corresponding to the abnormal pixel and the second X-ray tomographic image is outputted after the second image reconstruction unit 40 has completed reconstruction of the second X-ray tomographic image. According to this method, the first X-ray tomographic image is first displayed on the monitor 20 at a high speed in a short time period, then the second X-ray tomographic image having a higher image quality is automatically (unconditionally) displayed on the monitor 20.

Alternatively, when a given time period has elapsed from the completion of reconstruction of the second X-ray tomographic image, a switch may be made from the first X-ray tomographic image to the second X-ray tomographic image to output the selected X-ray tomographic image from the selector 50. According to this method, when two or more first X-ray tomographic images are successively displayed in a shorter time than a given time period, the second X-ray tomographic image is not displayed. On the other hand, when the user, interested,
allows the first X-ray tomographic image to be displayed longer than a given time period, the second X-ray tomographic image is automatically displayed.

Alternatively, after the completion of reconstruction of the second X-ray tomographic image, a switch may be made between the first X-ray tomographic image and the second X-ray tomographic image according to an instruction (switching instruction) given from the outside through the input unit 12 to output the selected X-ray tomographic image from the selector 50. According to this method, the user is allowed to selectively display the first X-ray tomographic image or the second X-ray tomographic image at any timing desired for any X-ray tomographic image.

After the completion of correction of the pixel data corresponding to the abnormal pixels in the first image correction unit 46, the first X-ray tomographic image is outputted from the selector 50 and the abnormal pixel data portions in the first X-ray tomographic image are sequentially replaced in real time by the corresponding abnormal pixel data portions in the reconstructed second X-ray tomographic image each time one abnormal pixel data portion of the second X-ray tomographic image (pixel data portion of the second X-ray tomographic image corresponding to the abnormal pixel data of the first X-ray tomographic image) is reconstructed in the second image reconstruction unit 40 until reconstruction of the second X-ray tomographic image is completed. According to this method, a switch is made from the first X-ray tomographic image to the second X-ray tomographic image for a given unit (the unit may be a pixel, a line of pixels, etc.) during reconstruction of the second X-ray tomographic image in lieu of making a switch from the first X-ray tomographic image to the second X-ray tomographic image after the completion of reconstruction of the second X-ray tomographic image. Thus, a high-definition X-ray tomographic image can be displayed by unit in order of reconstruction.

In addition, the second image reconstruction may be performed only for the pixels corresponding to the abnormal pixel data identified in the first image reconstruction. In this way, the pixels associated with the defective pixels are only reconstructed in the second image reconstruction, thus enabling the reconstruction at a higher speed.

Next, the operation of the imaging system 10 in tomosynthesis imaging will be described.

The subject 30 is positioned on the top surface of the imaging table 26, whereupon the input unit 12 gives instruction to start imaging, thereby starting tomosynthesis imaging controlled by the controller 14.

Upon start of imaging, the imager 16 irradiates the subject 30 with X-ray as the carrier moves the X-ray source 24 in one direction and changes the X-ray irradiation angle of the X-ray source 24 with respect to the subject 30 so that the subject 30 may be irradiated with X-ray at different imaging angles to acquire X-ray images taken at the different imaging angles during a single imaging operation. Each time an X-ray image of the subject 30 is acquired, the FPD 28 produces projection data corresponding to the X-ray image acquired.

The storage unit 32 of the image processor 18 stores the projection data of the X-ray images acquired by the imager 16.

Subsequently, the first image reconstruction unit 38 uses the projection data of the X-ray images taken at the different angles as stored in the storage unit 32 to perform reconstruction processing to thereby form an X-ray tomographic image at a cross section located at a given height of the subject 30.

Subsequently, the abnormal pixel data identification unit 42 refers to the defective pixel map to identify, from among the pixel data of the X-ray tomographic image reconstructed by the first image reconstruction unit 38, the pixel data of one or more abnormal pixels (abnormal pixel data) corresponding to one or more defective pixels on the FPD 28. Then, the first image correction unit 46 corrects the pixel data of the abnormal pixels in the X-ray tomographic image identified by the abnormal pixel data identification unit 42 to output a first X-ray tomographic image.

Concurrently with the processing for reconstructing the first X-ray tomographic image, the defective pixel data identification unit 44 refers to the defective pixel map to identify, from among the pixel data making up the projection data of the X-ray images taken at the different angles as stored in the storage unit 32, the pixel data corresponding to the defective pixels on the FPD 28, and the second image correction unit 48 corrects the pixel data corresponding to the identified defective pixels by interpolation using pixel data according to a well known method as described in, for example, JP 2007-632 A.

Then, the second image reconstruction unit 40 uses the projection data of the X-ray images corrected by the second image correction unit 48 to perform reconstruction processing to thereby form a second X-ray tomographic image at the cross section located at the same height of the subject 30 as does the first image reconstruction unit 38.

The selector 50 switches between the first X-ray tomographic image and the second X-ray tomographic image at a given timing according to the selection signal to output the selected X-ray tomographic image.

The X-ray tomographic image outputted from the selector 50 is displayed on the monitor 20. Controlled by the controller 14, the monitor 20 indicates information on the display status of the X-ray tomographic image (information showing which of the first and second X-ray tomographic images is displayed) and also displays an input screen for entering an instruction using the input unit 12 for selectively displaying the first X-ray tomographic image and the second X-ray tomographic image.

The user of the imaging system 10 can know by referring to the X-ray tomographic image display status information whether the X-ray tomographic image displayed on the monitor 20 is the first X-ray tomographic image or the second X-ray tomographic image. The user can freely switch between the first X-ray tomographic image and the second X-ray tomographic image at any timing desired by issuing a switching instruction through the instruction input screen from the input unit 12.

The X-ray tomographic image outputted from the selector 50 is supplied to the output unit 22, which, for example, prints out the X-ray tomographic image and allows digital image data of the X-ray tomographic image to be stored in a recording medium.

The imaging system 10 reconstructs the X-ray tomographic image, identifies the pixel data of the abnormal pixels in the X-ray tomographic image corresponding to the defective pixels on the FPD 28 and corrects the identified pixel data to reconstruct the X-ray tomographic image at a cross section located at a given height of the subject 30 and therefore the first X-ray tomographic image can be displayed at a higher speed in a shorter time period. A switch may also be made from the first X-ray tomographic image to the higher-definition second X-ray tomographic image. Alternatively, a switch may be made between the two images at any timing.

The specific structure of each component of the X-ray imaging system of the invention is not limited in any manner and may be achieved by any of various means used to provide similar functions.

The present invention is basically as described above.

The present invention, described above in detail, is not limited in any manner to the above embodiments and various improvements and modifications may be made without departing from the spirit of the invention.

1. An X-ray imaging system comprising:
an imager in which a subject is irradiated with X-ray at different angles while moving an X-ray source in one direction and the X-ray with which said subject has been irradiated is detected with a flat panel detector to acquire projection data of X-ray images taken at the different angles in tomosynthesis imaging;
a first image reconstruction unit in which the projection data of said X-ray images acquired by said imager is used to reconstruct an X-ray tomographic image;
an abnormal pixel data identification unit which refers to a defective pixel map in which information on defective pixels due to said flat panel detector and its peripheral circuit has been stored, to thereby identify, from among pixel data of the X-ray tomographic image reconstructed by said first image reconstruction unit, pixel data of abnormal pixels corresponding to said defective pixels; and
a first image correction unit which corrects the pixel data of the abnormal pixels of the X-ray tomographic image identified by said abnormal pixel data identification unit to output a first X-ray tomographic image.

2. The X-ray imaging system according to claim 1, wherein said abnormal pixel data identifying unit identifies the pixel data of the abnormal pixels of the X-ray tomographic image which extend linearly along a direction of movement of said X-ray source so as to correspond to said defective pixels, said first image correction unit performs defective pixel correction to the pixel data of the abnormal pixels of the X-ray tomographic image identified by said abnormal pixel data identification unit.

3. The X-ray imaging system according to claim 1, further comprising:
a defective pixel data identification unit which refers to said defective pixel map to identify, from among pixel data making up the projection data of the X-ray images acquired by said imager, pixel data corresponding to said defective pixels on said flat panel detector;
a second image correction unit which corrects each of the X-ray images acquired by said imager for the pixel data corresponding to the defective pixels identified by said defective pixel data identification unit;
a second image reconstruction unit which uses the projection data of the X-ray images corrected by said second image correction unit to reconstruct a second X-ray tomographic image; and
a selector which switches between said first X-ray tomographic image outputted from said first image correction unit and said second X-ray tomographic image reconstructed by said second image reconstruction unit at a given timing to selectively output said first X-ray tomographic image or said second X-ray tomographic image.

4. The X-ray imaging system according to claim 3, wherein said selector outputs said first X-ray tomographic image after said first image correction unit has completed correction of the pixel data corresponding to the abnormal pixels and outputs said second X-ray tomographic image after said second image reconstruction unit has completed reconstruction of said second X-ray tomographic image.

5. The X-ray imaging system according to claim 4, wherein after a predetermined time period has elapsed from completion of the reconstruction of said second X-ray tomographic image, said selector switches from said first X-ray tomographic image to said second X-ray tomographic image and outputs said second X-ray tomographic image.

6. The X-ray imaging system according to claim 3, wherein after reconstruction of said second X-ray tomographic image has been completed, said selector selectively outputs said first X-ray tomographic image or said second X-ray tomographic image according to an instruction entered from an outside.

7. The X-ray imaging system according to claim 3, wherein after said first image correction unit has completed correction of the pixel data corresponding to the abnormal pixels, said selector outputs said first X-ray tomographic image and abnormal pixel data portions in said first X-ray tomographic image are sequentially replaced by their corresponding abnormal pixel data portions in the reconstructed second X-ray tomographic image each time one abnormal pixel data portion of said second X-ray tomographic image is reconstructed in said second image reconstruction unit until reconstruction of said second X-ray tomographic image is completed.

8. The X-ray imaging system according to claim 3, further comprising:
an input unit for entering various instructions;
a controller for controlling operations of said X-ray imaging system according to an instruction entered from said input unit; and
a monitor for displaying said X-ray tomographic image outputted from said selector,
wherein said controller causes the monitor to indicate information on display status as to whether said first X-ray tomographic image is displayed or said second X-ray tomographic image is displayed.

9. The X-ray imaging system according to claim 8, wherein said controller causes said monitor to indicate an input screen for entering an instruction using said input unit for selectively displaying said first X-ray tomographic image or said second X-ray tomographic image.

10. The X-ray imaging system according to claim 3, wherein said second image reconstruction unit uses pixel data of abnormal pixels of said first X-ray tomographic image identified by said abnormal pixel data identification unit to reconstruct only pixels of said second X-ray tomographic image corresponding to the abnormal pixels of said first X-ray tomographic image.

11. An X-ray imaging method comprising the steps of:
irradiating a subject with X-ray at different angles while moving an X-ray source in one direction, and detecting the X-ray with which said subject has been irradiated with a flat panel detector to acquire projection data of X-ray images taken at the different angles in tomosynthesis imaging;
performing a first reconstruction in which the projection data of said acquired X-ray images is used to reconstruct an X-ray tomographic image;
performing a first identification for identifying, from among pixel data of the X-ray tomographic image reconstructed by said first image reconstruction, pixel data of abnormal pixels corresponding to defective pixels of said flat panel detector by reference to a defective pixel map in which information on the defective pixels due to said flat panel detector and its peripheral circuit has been stored; and
performing a first correction for correcting the pixel data of the abnormal pixels of the X-ray tomographic image identified by said first identification to output a first X-ray tomographic image.

12. The X-ray imaging method according to claim 11, further comprising the steps of:
performing a second identification for identifying, from among pixel data making up the projection data of the acquired X-ray images, the pixel data corresponding to the defective pixels in said flat panel detector;
performing a second correction for correcting the acquired X-ray images for the pixel data corresponding to the defective pixels identified by said second identification through interpolation using pixel data corresponding to pixels surrounding the defective pixels;
performing a second reconstruction for reconstructing a second X-ray tomographic image using the projection data of the X-ray images after said second correction has been performed; and
switching between said first X-ray tomographic image and said second X-ray tomographic image at a given timing to selectively output said first X-ray tomographic image or said second X-ray tomographic image.

13. A computer readable media including an X-ray imaging program for causing a computer to execute:
a step of receiving projection data of X-ray images taken at the different angles acquired by irradiating a subject with X-ray at different angles while moving an X-ray source in one direction and detecting the X-ray with which said subject has been irradiated with a flat panel detector in tomosynthesis imaging;
a step of performing a first reconstruction for reconstructing an X-ray tomographic image using the received projection data of the X-ray images;
a step of performing a first identification for identifying, from among pixel data of the X-ray tomographic image reconstructed by said first reconstruction, pixel data of abnormal pixels corresponding to defective pixels of said flat panel detector by reference to a defective pixel map in which information on the defective pixels due to said flat panel detector and its peripheral circuit has been stored; and
a step of subjecting the pixel data of the abnormal pixels of said X-ray tomographic image identified by said first identification to a first correction to output a first X-ray tomographic image.

14. A computer readable media including an X-ray imaging program for causing a computer to execute:
a step of receiving projection data of X-ray images taken at the different angles acquired by irradiating a subject with X-ray at different angles while moving an X-ray source in one direction and detecting the X-ray with which said subject has been irradiated with a flat panel detector in tomosynthesis imaging;
a step of performing a first reconstruction for reconstructing an X-ray tomographic image using the received projection data of the X-ray images;
a step of performing a first identification for identifying, from among pixel data of the X-ray tomographic image reconstructed by said first reconstruction, pixel data of abnormal pixels corresponding to defective pixels of said flat panel detector by reference to a defective pixel map in which information on the defective pixels due to said flat panel detector and its peripheral circuit has been stored; and
a step of subjecting the pixel data of the abnormal pixels of said X-ray tomographic image identified by said first identification to a first correction to output a first X-ray tomographic image:
for causing the computer to further execute:
a step of performing a second identification for identifying, from among pixel data making up the received projection data of the X-ray images, the pixel data corresponding to the defective pixels in said flat panel detector by reference to said defective pixel map;
a step of performing a second correction for correcting the received X-ray images for the pixel data corresponding to the defective pixels identified by said second identification through interpolation using pixel data corresponding to pixels surrounding said defective pixels;
a step of performing a second reconstruction for reconstructing a second X-ray tomographic image using the projection data of the X-ray images after said second correction has been performed; and
a step of switching between said first X-ray tomographic image and said second X-ray tomographic image at a given timing to selectively output said first X-ray tomographic image or said second X-ray tomographic image.
